(19) United States
(12) Patent Application Publication
(10) Pub. No.: US 2005/0061743 A1
(43) Pub. Date: Mar. 24, 2005

(54) Device and device for treating aqueous liquids in human medical treatment

(76) Inventor: Klaus Buttner, Klein Nordende (DE)

Correspondence Address: VENABLE, BAETJER, HOWARD AND CIVILETTI, LLP, P.O. BOX 34385, WASHINGTON, DC, 20043-9998, US

(21) Appl. No.: 10/493,022

(22) PCT Filed: Oct. 17, 2002
(86) PCT No.: PCT/EP02/11617

(30) Foreign Application Priority Data
Oct. 18, 2001 (DE) 101 51 488.3

(51) Int. Cl. B01D 61/24

(52) U.S. Cl. 210/646; 210/748; 250/437; 422/24

(57) ABSTRACT

The invention relates to a method for treating liquids, especially dialysis liquids, for purposes and for the elimination of harmful substances contained therein. The liquid is exposed to a UV radiation in the presence of physically dissolved oxygen, and is guided along the surface of a UV radiator in the form of a chaotic flow column or a thin surface film.

The invention relates to a method for treating liquids, especially dialysis liquids, for the purpose of sterilization and elimination of harmful substances contained therein, wherein the liquid is exposed to a UV radiation in the presence of physically dissolved oxygen.

For the purpose of human medical treatment where the patient directly or indirectly comes in contact with water or aqueous solutions, an extremely high purity of the water is an absolute necessity. However, an adequate water treatment is frequently not possible in practical operations because of a lack of technical means for realizing it. An adequate water treatment in this case refers to the elimination of problematic micro-organisms (germs, funguses, etc.) and pyrogenic agents (endotoxins, exotoxins), wherein harmful substances such as pesticides and the like must also be mentioned. This is particularly true for treatments requiring several liters of flowing aqueous liquids where the water for the treatment is drawn from the available municipal water supply without first being adequately treated. One example to be mentioned in this connection is the haemodialysis of patients suffering from kidney disease.

At the present time, the production of so-called “ultrapure water dialysate” for the above-described practical use is possible only when using a sterile filter in combination with an activated carbon filter. However, this treatment step is very cost-intensive and does not offer a satisfying solution over the long term due to the forming of multi-resistant germs. Even though physicians expressly demand ultrapure dialysate, it is not possible to provide it in every case at this point in time.

The theoretical options, known from literature, for UV-ray disinfection in principle provide alternatives to using a sterile filter for the purpose of treating the water for human medical treatments. It is known that the UV disinfection is suitable for sterilization as well as for producing so-called “ultrapure water” under certain conditions. Ultrapure water is used, for example, in the semiconductor industry and distinguishes itself by an extremely small share of carbon compounds besides having the properties of highly purified water. During the sterilization with UV rays, living micro-organisms are killed or inactivated by destroying the DNA with a wavelength of λ=254 nm. In addition, further harmful substances and endotoxins can be decomposed with the aid of UV rays and/or oxidative processes, which are triggered and maintained with the aid of ultrasound, in an interaction with the UV rays, such that they are no longer damaging to the human organism.

At the present time, no device or method based on the UV sterilization and oxidative processes generated in an interaction exists which makes it possible to produce ultrapure water. That is to say no device or method for killing micro-organisms, eliminating pyrogenic agents and decomposing harmful substances under dynamic conditions, meaning several seconds before the patient comes in direct or indirect contact with the water.

With the present invention, a device and a method have been developed for the first time for producing ultrapure water under dynamic conditions. The invention thus makes it possible to produce ultrapure water during a limited time interval that depends on the respective type of treatment, without requiring additional chemical additives or cost-intensive auxiliary means. As a result, these treatments are qualitatively raised to a noticeably higher level, which not only increases the quality of life for the patient, but in some circumstances can also be a life saver.

The present state of the technology does not disclose any device or method for solving this problem. Known methods only permit realizing several partial aspects of a water treatment, such as:
1. Sterilization of flowing liquids with UV rays of the wavelength λ=254 nm.
2. Decomposition of hydrocarbons in flowing liquids, using the wavelength λ=185 nm in combination with additional methods (no specifics from the manufacturer).
3. Sterilization and oxidation of organic substances with UV rays of the wavelength λ=254 nm and by adding OH⁻ radical generators such as hydrogen peroxide and ozone.
4. Generating of radicals through irradiating of titanium dioxide with λ=360 nm for disinfecting static systems such as surfaces.
5. Xenon lamps which are still in the developmental stage and are not yet commercially available for generating UV rays with a wavelength of λ=170 nm for cleaving H₂O into H and OH. The sterilizing effect of this wavelengths is no longer a given.

It is the object of the present invention to produce sterile and endotoxin-free water or aqueous solution under dynamic conditions with the aid of UV rays for use in human medical treatment and in the food industry. In particular, the object is to produce an “ultrapure dialysate,” having a flow rate ranging from 0 ml (static) to at least 10,000 ml/min and, preferably, for producing infusions with the online method.

The invention furthermore relates to a method for treating liquids, in particular dialysis liquids, for the purpose of sterilization and elimination of harmful substances contained therein, wherein the liquid is exposed to a UV radiation in the presence of physically dissolved oxygen and wherein the liquid is guided along the surface of a UV radiator in the form of a chaotic flow column or thin surface film.

The invention furthermore relates to a device for treating liquids, in particular dialysis liquids, for the purpose of sterilization and elimination of harmful substances contained therein. The device comprises a UV radiator and a case surrounding the UV radiator, wherein an additional internal case surrounding the UV radiator is also provided, which is designed to have openings at the top for allowing the liquid to flow from the outside to the inside, such that the liquid flows through the openings and onto the surface of the UV radiator to form a thin flow film thereon.

This object is solved above all with the following features:
1. Killing or inactivating of living micro-organisms and funguses in flowing water or aqueous solutions.
2. Elimination of pyrogenic agents (endotoxins and exotoxins) in flowing water or aqueous solutions.
3. Elimination of pesticides, herbicides and fungicides in flowing water or aqueous solutions.
4. Enriching of the dialysis liquid with oxygen to help boost the vitality of the dialysis patient during the dialysis treatment.

A device in which the following process sequences take place is required to achieve this goal:

The generating of ⁻OH radicals is initiated and maintained through the combination of selected UV wavelengths and/or an integrated agitating mechanism, preferably an ultrasound generator, wherein sufficient ⁻OH radicals are generated. These ⁻OH radicals are then distributed with sufficient uniformity in the liquid volume to ensure a high impact probability between ⁻OH radicals and endotoxins and/or exotoxins, wherein this is ensured under static and dynamic conditions with a flow rate of a few ml/min to at least 10,000 ml/min.

The device according to the invention essentially can comprise one or several hollow bodies through which the aqueous solution to be treated flows. It can be arranged outside of an apparatus or can be installed in an apparatus or can even form an integral part of an apparatus.

It is essential for the device according to the invention that at least one UV radiator is installed in the hollow body, wherein the radiator or radiators emits (emit) a suitable combination of at least two wavelengths from the spectrum 170 nm to 260 nm.

It is furthermore essential to have a chaotic flow of the liquid in the hollow body, around the UV radiator(s). This chaotic flow is preferably generated with an integrated ultrasound generator (frequency >18 kHz).

This ultrasound generator functions as a type of agitating mechanism as well as to produce H₂O₂. The UV radiator(s) in combination with an ultrasound generator emits (emit) at least one wavelength from the spectrum 170 to 260 nm.

Furthermore essential is a specific flow course for the medium to be treated inside the hollow body, relative to the radiator or the radiators, that is to say in a relatively thin layer on the glass surface of the UV radiator. The layer thickness of the aqueous solution to be cleaned depends on the turbulence, the contoured surface of the UV radiator and the chaos created in the liquid.

Also essential are the following parameters, which can be in relation to each other:

The hollow body diameter, the radiator tube diameter, the surface contour of the tube that is used, the inflow angle, the number of radiators, the outside surface contour of the protective tube for the radiator or the radiator screening tubes used, the UV wavelength, the radiation output, the radiation density, the dwell time for the medium to be sterilized, and the chaos generated in the aqueous solution in the radiated space.

Furthermore essential is a device installed inside the hollow body, which generates ⁻OH radicals and/or ozone and distributes these securely and uniformly in the flowing aqueous solution.

Also important is that the aqueous solution to be cleaned contains physically dissolved oxygen or that a device is present which feeds oxygen to the inside and/or into the flow chamber and obtains this oxygen either from the environmental air or oxygen supply containers—or even from the water itself.

The hollow body is provided with at least one inlet and at least one outlet for aqueous solutions. The openings can be at the bottom and/or at the top and can be installed either centered and/or tangentially aligned.

For the sterilization, the removal of endotoxins and the elimination of the hydrocarbons and/or aqueous solutions, the standard hose connection and/or tube connection is separated and the device according to the invention is then inserted as flow element with the aid of suitable couplings.

Depending on the tube diameter, the contour of the inside surface of the tube used, the inflow angle, the number of radiators, the outside surface of the radiator(s) used, the UV ray wavelength that is used, the radiation output, the radiation density, the chaotic state of the liquid molecules and the dwell time of the medium to be sterilized inside the tube, sterilization is securely achieved with a liquid throughput ranging from a few ml/min (static) to at least 10,000 ml/min when using the device according to the invention.

The invention is based on the production and secure distribution of ⁻OH radicals and/or ozone in water or aqueous solutions, from which the germs, pyrogenic agents (endotoxins, exotoxins) and hydrocarbons (fertilizers, pesticides, fungicides, herbicides) are removed. The following features are critical for this:
1. One or several UV burners emitting a specific wavelength or a suitable combination of at least one wavelength within the limit values of between 170 nm and 260 nm;
2. A device for generating ⁻OH radicals and/or ozone inside the hollow body, in the aqueous liquid to be treated;
3. A flow guidance which ensures that all regions of the liquid come in contact with OH radicals and/or
4. A suitable device which securely distributes the generated ⁻OH radicals in the flowing aqueous solution.

Possible embodiments are:
To 1: It is essential to have a combination of at least one UV wavelength within the limit values between 170 nm and 260 nm and a device on the inside of the case that ensures a turbulent flow and chaos in the aqueous solution to be cleaned.

To 1 and 2:
The following physical chemical reaction is triggered and maintained in the aqueous solution to be treated as a result of the correct selection of the wavelengths, the liquid guidance and the oxygen partial pressure in the water or the aqueous solution:
   Reaction I:
O₂+hv->0+O
O₂+O->O₃
   Reaction II:
O₃+hv->O₂+O
O+H₂O->H₂O₂
   Reaction III: H₂O₂+hv->OH+⁻OH
   Reaction IV: endotoxins+⁻OH
   →H₂O+CO₂+decomposition products

To 2:
glucose oxidase+glucose+oxygen
→gluconic acid+hydrogen peroxide

To 2:
Generating ⁻OH radicals from H₂O with a suitable UV wavelength
   →H₂O+hv H+⁻OH

To 4 and 2:
Hydrogen peroxide and/or peroxide acetic acid is introduced finely dosed into the liquid flow.

To 3:

The absorption in water of at least one of the UV wavelengths from the spectrum 170 nm to 260 nm is very high. To ensure that the complete liquid flow is confronted sufficiently with ⁻OH radicals, a flow guidance is necessary which ensures that the layer thickness for the water or the aqueous solution to be treated does not exceed the depth at which the UV rays used with the aforementioned wavelength range can penetrate in water or an aqueous liquid and/or a geometry is necessary that ensures a secure distribution of the generated ⁻OH radicals.

And/or an agitating mechanism that ensure chaos in the aqueous solution.

To 4:

The absorption in water of at least one of the UV wavelengths from the spectrum 170 nm to 260 nm is very high. To ensure that the complete liquid flow is confronted sufficiently with ⁻OH radicals, it must be ensured that at least one of the wavelengths from the spectrum 170 nm to 260 nm, required for generating ⁻OH radicals, sweeps over the complete flow of liquid. According to the invention, this is achieved by structuring the UV burner surface around which the liquid flows, or its protective tube, in the manner of stalactites or stalagmites. The surface can also be provided with rotation-symmetrical raised areas. Finally, it is possible to provide an agitating mechanism that ensures chaotic conditions in the aqueous solution.

To 5:

As a result of a suitable correlation of the following features: tube diameter, inside surface contour of the tube used, inflow angle, number of radiators, outside surface of the radiator(s) used, UV wavelength, radiation output, radiation density, dwell time of the medium to be sterilized in the tube and the chaos created therein, sterility and the state of being free of endotoxins can be securely achieved with a flow-through rate between 0 ml/mn (static) and at least 10,000 ml/min.

The above-described device for creating sterile and endotoxin-free water or aqueous solutions is particularly suitable for ensuring a more secure environment for patients and personnel during a haemodialysis treatment. That is to say, the haemodialysis treatment as described in the following positions can be carried out with higher safety by the treatment specialist while also being more effective and cheaper.

1. Online Dialysis Method:

During the realization of specific haemodialysis treatments, a considerably higher amount of liquid (substitute) is withdrawn from the patient than can be continuously replenished from the patient's liquid potential. The online dialysis has been and in part still is carried out with the aid of substitute liquids purchased in bags and supplied to the patient via compensation devices. In the process, a portion of the dialysis liquid produced by the dialysis machine is cleaned with the aid of sterile filters, such that it can be used as substitute, a process that is very expensive. In addition, the substitute is produced from non-sterile dialysis liquid containing pyrogenic agents and endotoxins-/exotoxins. It means that the patient's safety is ensured only if all capillaries of the sterile filter used are without micro-leaks.

2. Safety of the Patient:

The patient is subjected to considerable stress during the dialysis treatment as a result of the non-sterile dialysis liquid. Furthermore, the Venturi principle is used with nearly all dialysis machines on the market for de-gassing the dialysis liquid with the effluent-water flow, the dialysate. In the process, a retrograde germ development can occur in the dialysis liquid. Sterile filters have a relatively short service life of approximately 200 h of dialysis operation. That is to say, the sterile filter must be replaced at least every two months. No indicator is available at this time to indicate the depletion of the filter. This results in a monitoring problem and additionally represents a danger source during the filter replacement because the micro-organisms to be eliminated are not killed but accumulate inside the filter. For that reason, the filter also seems to contribute to the generating of multi-resistant germs. Not least, micro-leaks can also develop as a result of capillary breaks and can function as passages for micro-organisms and thus can represent an enormous danger to the patient.

3. Danger to the Personnel:

For cost reasons, sterile filters are presently used only in front of the dialysis machine if they are used at all. It means that the dialysis machine is unprotected in the region for the dialysis liquid as well as in the effluent water region (dialysate). The dialysis machine thus offers optimum growth conditions for any germs that enter. The accumulation of germs represent a considerable source of danger to the technical personnel. The filter replacement can also be a source of danger.

4. Environmental Protection and Hygiene:

So far, used dialysis liquid, the dialysate, has been released into the waste-water systems without being cleaned. As a result of the considerable protein deposits in the waste-water pipe system, this represented a fundamental problem for dialysis operators. These deposits form an excellent nutrient medium for all micro-organisms and are particularly critical if highly infectious (hepatitis, HIV, etc.) dialysis patients are treated.

Remedial Actions According to the Invention:

First of all, the online dialysis can be realized more securely in that the substitute is produced from already sterile dialysis liquid, which is then sterilized again and depleted of endotoxins. It becomes even safer in that the micro-organisms are not raised to a different matrix, but are killed and their fragments essentially oxidized to H₂O and CO₂. In addition, no micro-leak can develop due to the breakage of filter capillaries.

The permeate, the dialysis liquid, the dialysate, as well as the substitution solution are sterilized and cleaned of endotoxins, thus precluding the dangers listed in the following:
a) Stress to the patient caused by non-sterile and endotoxin-loaded dialysate;
b) Retrograde germ development during the de-gassing of the dialysis liquid while the used dialysis liquid (dialysate) is sterilized;
c) No development of multi-resistant germs because all micro-organisms are killed and their fragments are essentially oxidized to H₂O and CO₂.

The permeate, the dialysis liquid, the dialysate, as well as the substitution solution are sterilized and cleaned of endotoxins, thus precluding the dangers described in the following and achieving the advantages listed below:
a) A retrograde germ development is impossible during the de-gassing of the dialysis liquid because the used dialysis liquid (dialysate) is sterilized, thus precluding the risk of contamination and the danger of infection to the service technician.
b) The considerably lower number of service intervals (12,000 h or approximately 4.5 years dialysis operation) reduces the operating time and the danger of secondary contamination.
c) The efficiency can be monitored through reading of the indicator.
d) No special solid-waste disposal of a filter loaded with micro-organisms is required since the micro-organisms are killed.
e) There is no longer any contamination caused by deposits etc, something that basically cannot be avoided when liquid flows through pipes. According to the invention a self-cleaning system is created as a result of the ultrasound agitating mechanism.

The invention is explained in the following with the aid of examples and the drawing, showing in:

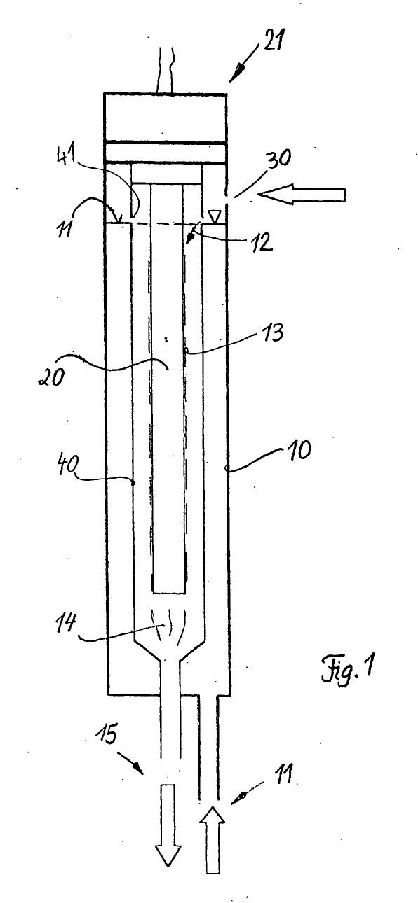

FIG. 1 A cross-sectional view through a device according to the invention.

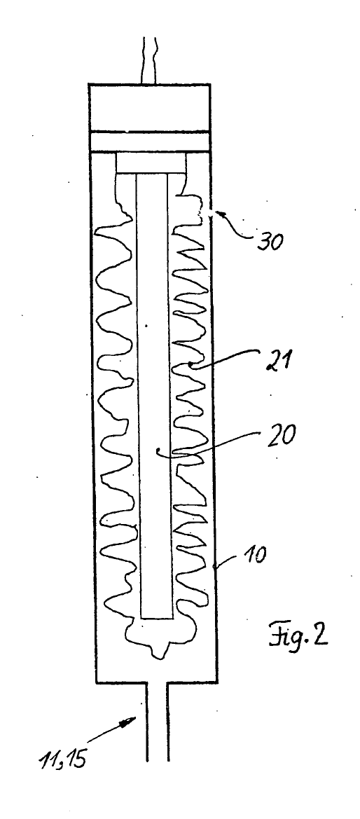

FIG. 2 A corresponding cross-sectional view of a modified embodiment of the device according to the invention.

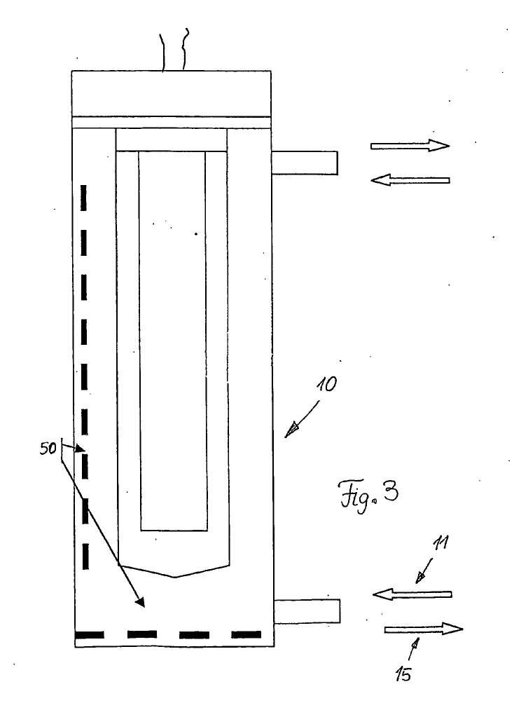

FIG. 3 A modified embodiment of the device according to the invention.

The embodiments shown in FIGS. 1 to 3 essentially have a rotation-symmetrical design, wherein the individual case parts are made of glass.

Reference number 10 refers to a cylindrical outer case into which the liquid to be treated is fed from below at point 11. The liquid can be pumped in with the aid of a pump and throttle valve, so that a specific volume per time unit flows into the case 10. A liquid level 11 then adjusts in the top region of case 10.

The cylindrical case 10 encloses an additional cylindrical case 40 on the inside which is tapered in the lower region and is connected to a line extending out from the case 10 at a sealed location. In the upper region, the internal cylinder 40 is provided with openings 41 at the same height, wherein FIG. 1 shows two opposite arranged openings 41. These openings or holes can be slot-shaped and ensure that the level 11 adjusts for the aqueous liquid inside the cylinder 10. At point 12, an arrow indicates how this liquid enters the inside space of cylinder 40 and, in the process, forms a relatively thin surface layer on the outside of a UV radiator casing. The liquid runs down on the case 20 and leaves the case at point 14 to flow via the above-mentioned pipe at point 15 into a catch basin.

The external case 10 is provided with a connecting passage at point 30, which allows the environmental air to enter the inside space of the container 10, so that the oxygen in the air comes in contact with the liquid to be treated. For the embodiment shown herein, the environmental air flows through the opening 30 into the container 10. For one preferred embodiment, oxygen can be supplied instead.

Reference 21 finally refers to the UV radiator mount which is supplied on the one hand with electrical energy while on the other hand it also functions as mechanical holding device for the cylinders 10 and 40.

The liquid to be treated flows at 11 into the case 10 and finally forms a relatively thin layer or a film 13 on the surface of the UV radiator 20. If the UV radiator is turned on, this film is exposed to the respective radiation. As a result of the relatively thin flow film, the liquid flow can be treated uniformly the UV radiator 20, using the wavelengths in question, so as to achieve the desired effect which involves the generating of OH⁻ radicals with the aid of physically dissolved oxygen or, as previously described, in the case of liquids that do not contain physically dissolved oxygen.

FIG. 2 shows a modified embodiment of the device according to the invention where the outside contour 21 of the UV radiator 20 is designed such that the flowing liquid must cover a relatively long distance during which it is subjected to intensive radiation, in particular a radiation having the wavelength of 185 nm. This radiation affects the liquid 1 only over a short distance from the UV radiator surface since absorption processes occur with higher thicknesses of the liquid film and counteract the generating of OH— radicals. This effect of the larger UV radiator surface and/or the longer distance traveled by the liquid can be further increased by installing obstacles in the flow path which create turbulences in the liquid.

Within the framework of the present invention, devices of the type as shown in FIGS. 1 and 2 can also be installed one behind the other, so that the desired treatment of the liquid can occur over several successive stages.

The inside wall of the outer container furthermore can conceivably have a non-cylindrical surface, e.g. a surface that corresponds to the surface 21 of the UV radiator, so that the liquid must flow through relatively narrow flow passages. An easy to realize shape of this type would be a helical or screw-type surface for the UV radiator 20 and a matching but slightly larger inside surface for the case 40.

It is furthermore possible within the framework of the present invention to reverse the flow direction of the liquid, shown in FIGS. 1 and 2, so that the liquid column flows from the bottom toward the top along the burner case.

FIG. 3 shows several ultrasound transmitters 50 on the side wall or in the lower region of the case 10. However, a single ultrasound rod that extends parallel to the UV burner 20 could also be used.

1. A device for the treatment of liquids, in particular dialysis liquids for the purpose of sterilization and elimination of harmful substances contained therein, said device comprising a UV radiator and a case surrounding the UV radiator, characterized in that an additional inside case (4) that surrounds the UV radiator (20) is arranged inside the case (10), that the inside case (40) is designed to have openings (41) at the top for allowing the liquid to flow from the outside via the openings (41) to the inside to be guided onto the surface of the UV radiator (20), such that a thin flow film (13) forms thereon and that an ultrasound probe is provided for generating and maintaining a chaotic flow along the UV burner or the UV burners and for producing H₂O.

2. A device for treating liquids, in particular dialysis liquids for the purpose of sterilization and elimination of harmful substances contained therein, said device comprising a UV radiator and a case surrounding the UV radiator, characterized in that an additional inside case (40) that surrounds the UV radiator (20) is arranged inside the case (10), that the inside case (40) is designed to have openings (41) at the top for allowing the liquid to flow from the outside via the openings (41) to the inside to be guided onto the surface of the UV radiator (20), such that a thin flow film (13) forms thereon.

3. The device with ultrasound probe for generating and maintaining a chaotic flow along the UV burner or the UV burners and for producing H₂O₂.

4. The device according to claim 2, characterized in that the surface of the UV radiator (20) is a surface (21) that is enlarged past the standard inside jacket surface.

5. The device according to claim 1, characterized in that it is arranged in a liquid dialysis flow circulation for a haemodialysis machine.
